(19) United States
(12) Patent Application Publication
(10) Pub. No.: US 2023/0123238 A1
(43) Pub. Date: Apr. 20, 2023

(54) CUSTOMIZED NIPPLES FOR BABY AND MEDICAL PRODUCTS BASED ON ENHANCED ANALYSIS OF SCANNED NIPPLES

(71) Applicants: Cassandra F. Bolten, Poway, CA (US); Ellen Zuckerman, Portland, OR (US); David A. Followell, Wildwood, MO (US)

(72) Inventors: Cassandra F. Bolten, Poway, CA (US); Ellen Zuckerman, Portland, OR (US); David A. Followell, Wildwood, MO (US)

(21) Appl. No.: 17/954,324

(22) Filed: Sep. 28, 2022

Related U.S. Application Data
(60) Provisional application No. 63/249,827, filed on Sep. 29, 2021.

(51) Int. Cl. A61J 11/00; G06F 30/27; G06F 30/10

(52) U.S. Cl. CPC A61J 11/005; G06F 30/10; G06F 30/27; A61B 5/4312

(57) ABSTRACT

Customized nipples are provided herein that are designed to mimic a woman’s anatomic nipple to improve experiences such as breast feeding, soothing a baby, and/or superior aesthetics. The customized nipples are created in a noninvasive manner, for example, via a scan of the woman’s breast that is received by a computer. The scan may be processed with enhanced computing power to provide the optimal customized nipple for the woman. The customized nipples may be formed for baby/nipple/nursing products such as bottles, pacifiers, nipple shields, breast pump flanges, attachments for supplemental nursing systems, and prosthetic nipples.

### CROSS-REFERENCE TO RELATED APPLICATIONS

This application claims priority to U.S. Provisional Pat. Application No. 63/249,827, filed Sep. 29, 2021, the entire contents of which are incorporated herein by reference.

### TECHNICAL FIELD

This technology generally relates to customized nipples for baby/nipple/nursing products such as bottles, pacifiers, nipple shields, breast pump flanges, attachments for supplemental nursing systems, and prosthetic nipples, along with efficient methods of making the same using enhanced analysis of scanned nipples.

### BACKGROUND

There are a number of baby bottles and pacifiers currently on the market. Oftentimes, especially in the first few months in a baby’s life, a nursing woman is counseled that use of a pacifier or bottle would be unwise as the baby may develop “nipple confusion” from the pacifier/bottle and thereafter reject the woman’s breast for nursing. Accordingly, there is a need for a nipple designed to minimize the risks of such nipple confusion.

U.S. Pat. No. 9,044,379 to Sabree describes a customized nipple using a mold to create a replica of the mother’s breast. The nipple in Sabree suffers from a number of drawbacks in that a physical mold of the woman’s breast is required. In practice, however, women would generally prefer that a physical mold not be required for making the nipple replica because of the physical contact to the breast, along with time and cost associated with this complex approach.

U.S. Pat. No. 5,108,686 to Griffin and U.S. Pat. App. Pub. No. 2008/0616776 to Cooke are further examples of customized nipples, yet both these examples also require a mold for forming the customized nipple.

Design Continuum proposed a customized nipple design in a socket wrench-like bottle that simulated the mother’s breast. Such a design was criticized and never appeared to make it to market. The design suffered from drawbacks such as incompatibility with practical bottle designs.

U.S. Pat. App. Pub. No. 2017/0614186 to Lofaro describes an artificial nipple that is formed of an elastomeric polymer by use of 3D printing after imaging of a mother’s breast in an active state of lactation.

Additional nipple-related products available on the market are nipple shields, breast pump flanges, and prosthetic nipples. Nipple shields are typically used to assist a nursing mother with breast feeding including as a component of supplemental nursing systems, while breast pump flanges are coupled to a breast pump and directly contact the nursing mother’s breasts. Prosthetic nipples may be used in a medical setting, such as following a mastectomy or other trauma. Such nipple-related products are not based on the woman’s specific anatomy, resulting is discomfort and/or emotional tolls.

U.S. Pat. App. Pub. No. 2019/0291307 to Wright describes a process of 3D scanning lactating women’s breasts to generate an AutoCAD model of the maternal nipple.

There is a need for a nipple for baby/nipple/nursing products that is customized to the woman’s actual nipple and formed in a noninvasive, efficient manner.

U.S. Pat. No. 10,449,121 to Bolten, the entire contents of which are incorporated herein by reference, solved many of the drawbacks described above by providing customized nipples designed to mimic a woman’s anatomic nipple based on a scan.

### SUMMARY

Customized nipples are provided herein that are designed to mimic a woman’s anatomic nipple for benefits such as reducing nipple confusion for a nursing baby, reducing discomfort for the woman, and/or aesthetics. The customized nipples are created in a noninvasive manner, for example, via a scan of the woman’s breast that is received by a computer. The customized nipples are formed in an efficient manner, preferably, within a short time period after the scan or pre-formed and matched responsive to the scan. The customized nipples may be formed using a device, e.g., a three-dimensional printer, located in the same location as the scanner for fast nipple replication. Advantageously, in such instances, the woman’s nipple (along with the areola and/or breast, if desired) may be scanned and the customized nipple formed in a single location in a relatively short amount of time (e.g., less than one hour). For example, the customized nipple system may be located in a hospital or doctor’s office where a woman can conveniently be scanned and receive the customized nipple in a single visit, as well as at home or during a meeting with a lactation consultant. The system could also be mobile, e.g., on a movable cart, that could be moved to the woman. As another example, aspects on the system could be embodied in software downloaded to a computer that utilizes components of the computer for aspects of the scanning and processing the customized nipple described herein. The customized nipples are preferably formed for baby/nipple/nursing products such as bottles, pacifiers, nipple shields, breast pump flanges, attachments for supplemental nursing systems, and prosthetic nipples.

In accordance with one aspect of the present invention, systems and methods are provided for forming a customized nipple for use in a baby/nipple/nursing product. A scan of a woman’s nipple may be used to generate data indicative of characteristics of the woman’s nipple. The scan of the nipple may also include scans of the woman’s areola, breast, etc. The data may be processed to compare the characteristics of the woman’s nipple to a plurality of other nipples’ characteristics. Information may be generated for a customized nipple for the woman based on the comparison of the characteristics of the woman’s nipple with the other nipples’ characteristics such that the customized nipple mimics the characteristics of the woman’s nipple.

The characteristics of the woman’s nipple may be diameter of the nipple, diameter of an areola, depth/length of the nipple, depth/length of the areola, depth/length of the nipple plus the areola, color of the nipple, color of the areola, slope of the nipple, slope of the areola, texture of the nipple, or texture of the areola, or any combination thereof.

Processing the data may include generating a score for each of the characteristics of the woman’s nipple to compare to corresponding scores stored for the plurality of other nipples’ characteristics. The scores may be weighted by characteristic such that scores of a more significant characteristic receive a higher weighted value than less significant characteristics. The scores may be represented for each of the characteristics as at least one vector. The vector(s) may be used to generate a matrix incorporating multiple vectors associated with the characteristics. For example, the vector(s) may be inputted into at least one neural network to generate at least one matrix for the customized nipple for the woman. The systems and methods described herein may utilize machine learning/artificial intelligence technology for generating the products described herein. For example, the at least one neural network utilizes machine learning technology for the customized nipple of the woman. Processing the data may include classifying characteristics of the woman’s nipple. The characteristics may be identified by machine classification. The characteristics may be predetermined based on a classified dataset of the plurality of other nipples’ characteristics. The systems and methods may train a trained model of classified datasets associated with characteristics of the plurality of other nipples using the classified characteristics of the woman’s nipple. Such training is expected to improve classification of the nipple/breast scans for optimal product selection/formation.

The systems and methods may be performed using an “app” downloaded to a computing device. The data may be sent to a remote computing system and processing the data and generating the information may occur at the remote computing system. The systems and methods may further cause at least one product incorporating the customized nipple to be sent to the woman. The product(s) may be, for example, a baby bottle nipple, pacifier, prosthetic nipple, nipple shield, attachment for supplemental nursing system, or breast pump flange, or any combination thereof.

Generating the information may include selecting the customized nipple for the woman from a plurality of customized nipples with predetermined characteristics, each of the plurality of customized nipples having at least one different predetermined characteristic from another. Selecting the customized nipple may include executing a matching algorithm to match the woman’s nipple to the customized nipple. The customized nipple may be formed. For example, the customized nipple may be formed as part of a complete product(s) or as a component to be incorporated into the product(s). Further, forming the customized nipple may include forming portions also including customized areola and/or breast characteristics. In accordance with some embodiments, forming the customized nipple includes printing (e.g., 3D printing) the customized nipple.

The systems and methods may include receiving user input associated with the customized nipple at a user interface. The customized nipple may be formed of a material selected to mimic feel of the woman’s nipple. The material may be selected from silicone, rubber, poly vinyl chloride, or latex, or combinations thereof. The systems and methods may include scaling the customized nipple based on user input associated with information on the woman’s baby.

A bottle is provided herein that includes the customized nipple. A pacifier is provided herein that includes the customized nipple. A nipple shield is provided herein that includes the customized nipple. A prosthetic nipple is provided herein that includes the customized nipple. A breast pump flange is provided herein that includes the customized nipple. A component for a supplemental nursing system (SNS) is provided herein that includes the customized nipple.

In accordance with some aspects, a non-transitory computer readable medium comprising instructions that, when executed by at least one processor, cause the at least one processor to: receive data indicative of characteristics of a woman’s nipple based on a scan of the woman’s nipple; process the data to compare the characteristics of the woman’s nipple to a plurality of other nipples’ characteristics; and generate information for a customized nipple for the woman based on the comparison of the characteristics of the woman’s nipple with the other nipples’ characteristics such that the customized nipple mimics the characteristics of the woman’s nipple.

In accordance with other aspects, a system for generating information associated with a customized nipple for use in a baby or nursing or nipple-related product is provided herein. The system may include at least one processor configured to execute instructions to cause the at least one processor to: receive data indicative of characteristics of a woman’s nipple based on a scan of the woman’s nipple; process the data to compare the characteristics of the woman’s nipple to a plurality of other nipples’ characteristics; and generate information for a customized nipple for the woman based on the comparison of the characteristics of the woman’s nipple with the other nipples’ characteristics such that the customized nipple mimics the characteristics of the woman’s nipple.

### BRIEF DESCRIPTION OF THE DRAWINGS

FIG. 1A shows an embodiment of an exemplary customized nipple on a bottle while FIG. 1B shows the exemplary customized nipple of FIG. 1A constructed in accordance with the principles of the present invention.

FIG. 1C is a cross-sectional view of the exemplary customized nipple of FIG. 1B.

FIG. 2 shows an embodiment of an exemplary customized nipple on a pacifier constructed in accordance with the principles of the present invention.

FIG. 3 shows an embodiment of an exemplary customized nipple for a nipple shield and/or an attachment to a supplemental nursing system constructed in accordance with the principles of the present invention.

FIG. 4 shows an embodiment of an exemplary customized nipple on a prosthetic nipple constructed in accordance with the principles of the present invention.

FIG. 5 shows an embodiment of an exemplary customized nipple on a breast pump flange constructed in accordance with the principles of the present invention.

FIG. 6 shows an embodiment of an exemplary system for processing information for a customized nipple in accordance with the principles of the present invention.

FIG. 7 is a schematic view of exemplary hardware and software components of an exemplary computing system in accordance with the principles of the present invention.

FIG. 8 shows an embodiment of components of an exemplary computing system for processing information for a customized nipple in accordance with the principles of the present invention that may include, for example, machine learning technology.

FIG. 9 shows an embodiment of an exemplary method for processing information for a customized nipple and, optionally, for enhanced analysis of scanned nipples in accordance with the principles of the present invention.

### DETAILED DESCRIPTION OF THE INVENTION

Customized nipples are provided herein that are designed to mimic a woman’s anatomic nipple for beneficial results such as minimizing nipple confusion for a nursing baby, minimizing discomfort for a nursing mother during breast feeding and/or pumping, and/or for superior aesthetics. The customized nipples are created in a noninvasive manner, for example, via a scan of the woman’s breast that is received by a computer. The customized nipples also are preferably formed in an efficient manner within a short time period after the scan. The customized nipples may be matched to a preferred customized nipple of a plurality of predetermined nipples based on characteristics of the nipple determined from the scan. The woman’s nipple may be scanned and the customized nipple formed in a single location. The scanned results may be compared to prior scanned results to determine the preferred customized nipple using, for example, machine learning technology incorporating a trained model of classified nipple characteristics. The customized nipples are preferably formed for baby/nipple/nursing products such as bottles, pacifiers, nipple shields, breast pump flanges, attachments for supplemental nursing systems, and prosthetic nipples.

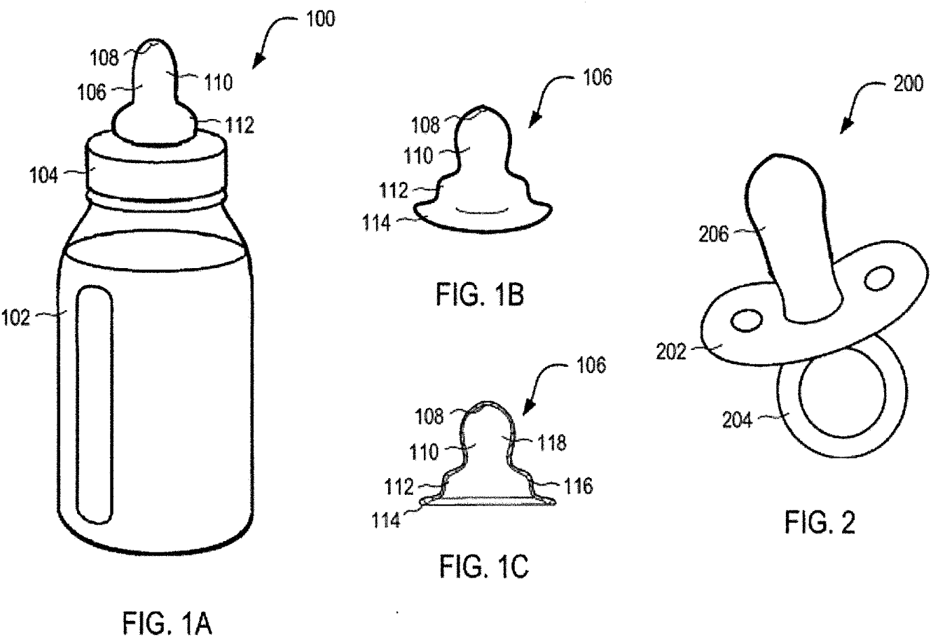

Referring to FIG. 1A, an exemplary customized nipple on a bottle is shown. Bottle 100 illustratively includes container 102, cap 104, and customized nipple 106. Container 102 holds fluid such as breast milk. Cap 104 is removably coupled to container 102, for example, via corresponding threads. Cap 104 has an aperture to receive customized nipple 106 therethrough. Container 102 and cap 104 may be a commercially available baby bottle such as Dr. Brown’s Natural Flow Standard bottles from Handi-Craft Company of St. Louis, Missouri or of U.S. Pat. No. 9,486,392 to Kemper, the entire contents of which are incorporated herein by reference, or Evenflo classic or advanced angled bottles of Evenflo Feeding, Inc. of West Chester, Ohio. As will be readily apparent to one skilled in the art, bottle 100 may have additional components such as anti-colic tubing.

Customized nipple 106 is sized and shaped to mimic a nursing woman’s anatomic nipple to minimize nipple confusion for a nursing baby. Customized nipple 106 may be constructed to be interchangeable with the nipple of a commercially available bottle. For example, a user may select the type of bottle for use with customized nipple 106 and the systems described herein will construct the nipple to fit to the bottle based on data stored in memory of the systems. Customized nipple 106 is formed of a material selected to mimic feel of the nursing woman’s nipple such as silicone, rubber, poly vinyl chloride, or latex, or combinations thereof. In embodiments where customized nipple 106 is designed for use in a bottle, customized nipple 106 has hole 108 through which the baby can suck fluid, e.g., breast milk. The size of hole 108 may be selected by the user based on the baby’s perceived preference. Hole sizes known in the art such as size 1, 2, 3, 4, 5, etc. may be selected and the hole size formed based on the selection. Illustratively, customized nipple 106 has mimic nipple portion 110 and intermediate portion 112. Mimic nipple portion 110 mimics the size and shape of the nursing woman’s nipple based on the scan as described herein. Intermediate portion 112, which could also be formed based on the scan, has an outer diameter greater than the greatest outer diameter of mimic nipple portion 110, but smaller than the inner diameter of the aperture of cap 104 to permit cap 104 to receive customized nipple 106 through the aperture although a portion of customized nipple 106 remains between cap 104 and container 102.

FIG. 1B illustrates an exemplary standalone customized nipple 106 formed in accordance with the principles of the present invention. Customized nipple 106 has mimic nipple portion 110 and intermediate portion 112 as well as base ring 114 to maintain customized nipple 106 in cap 104 when bottle 100 is assembled. Base ring 114 has an outer diameter greater than the inner diameter of the aperture of cap 104. Mimic nipple portion 110, intermediate portion 112, and base ring 114 may be integrally formed from a common material. Additionally, customized nipple 106 may be a standalone unit that is interchangeable with a plurality of bottles. As shown, the longitudinal axis of mimic nipple portion 110 is aligned with a longitudinal axis of container 102 for usability and interchangeability. The longitudinal axis of container 102 may extend through the full length of container 102 or may extend through part of the length of container 102, for example, when a portion of container 102 is angled at a predetermined angle such as 45 degrees.

FIG. 1C is a cross-sectional view of customized nipple 106 of FIG. 1B. Wall 116 of customized nipple 106 defines cavity 118 such that fluid can travel from container 102 into cavity 118 and out of hole 108 for consumption. Wall 116 may have a generally uniform thickness although the thickness can vary.

Referring now to FIG. 2, an exemplary customized nipple on a pacifier is shown. Pacifier 200 illustratively includes pacifier base 202, handle 204, and customized nipple 206. Pacifier base 202 is coupled to, or integrally formed with, handle 204 and/or customized nipple 206. Pacifier base 202 is a support structure for customized nipple 206.

Customized nipple 206 is sized and shaped to mimic a nursing woman’s anatomic nipple to minimize nipple confusion for a nursing baby. Customized nipple 206 may be constructed to be formed on a commercially available pacifier base with a handle. For example, a user may select the type of pacifier for use with customized nipple 206 and the systems described herein will construct the nipple on a commercially available pacifier base with a handle or may construct pacifier base 202 and handle 204 based on data stored in memory of the systems and construct customized nipple 206 based on the scanned information of the nursing woman’s nipple. In this manner, customized nipple 206 may be formed on a plurality of different types of pacifier bases. Customized nipple 206 is formed of a material selected to mimic feel of the nursing woman’s nipple such as silicone, rubber, poly vinyl chloride, or latex, or combinations thereof. Customized nipple 206 of pacifier 200 may be hollow or may be solid.

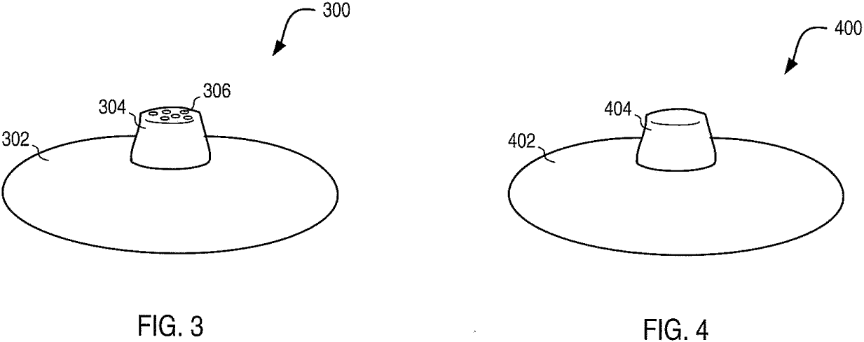

Referring now to FIG. 3, an exemplary customized nipple is shown for use in a nipple shield and/or an attachment to a supplemental nursing system. Device 300 illustratively includes shield base 302, customized nipple 304, and aperture(s) 306. Shield base 302 is coupled to, or integrally formed with, customized nipple 304. Shield base 302 may include an adhesive on the inner surface to assist with adhering nipple shield 300 to the breast. One or more apertures 306 are preferably included in customize nipple 304, for example, at the end surface as shown. Apertures 306 permit breast milk to travel therethrough when the nursing woman’s breast is stimulated, e.g., by latching of the baby.

Customized nipple 304 is sized and shaped to mimic a nursing woman’s anatomic nipple to reduce discomfort to the nursing woman and/or nipple confusion for a nursing baby. Customized nipple 304 may be constructed to be formed on a commercially available nipple shield base or together with other commercially available supplemental nursing system components. Alternatively, shield base 302 may be constructed based on data stored in memory of the systems while customized nipple 304 is constructed based on the scanned information of the nursing woman’s nipple, wherein the scanned information may include scans of the woman’s areola and/or breast. Customized nipple 304 is formed of a material commonly used in nipple shields/supplemental nursing systems such as a polymer and/or a material selected to mimic feel of the nursing woman’s nipple such as silicone, rubber, poly vinyl chloride, or latex, or combinations thereof.

Referring now to FIG. 4, an exemplary customized nipple on a prosthetic nipple is shown. Prosthetic nipple 400 illustratively includes customized areola 402 and customized nipple 404. Customized areola 402 is coupled to, or integrally formed with, customized nipple 404. Customized areola 402 may include an adhesive on the inner surface to assist with adhering prosthetic nipple 400 to the breast. Customized nipple 404 is sized and shaped to mimic a woman’s anatomic nipple and customized areola 402 is sized and shaped to mimic a woman’s anatomic areola for aesthetics and/or emotional reasons. Customized areola 402 and customized nipple 404 are formed of a material selected to mimic feel of the woman’s nipple such as silicone, rubber, poly vinyl chloride, or latex, or combinations thereof. In the case of a mastectomy, the anatomical nipple/areola is preferably scanned before the mastectomy. Alternatively, in the case of a single mastectomy, the remaining anatomical nipple/areola may be scanned for forming prosthetic nipple 400.

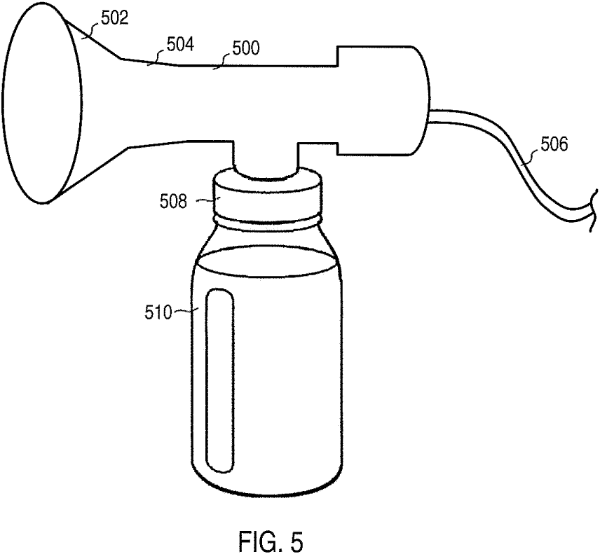

Referring now to FIG. 5, an exemplary customized nipple on a breast pump flange is shown. Breast pump flange 500 illustratively includes breast flange 502, customized nipple portion 504, tube 506, and/or bottle connector 508. Breast flange 502 is coupled to, or integrally formed with, customized nipple 504. Breast flange 502 is shaped to receive a breast for pumping milk therefrom. Breast flange 502 may be formed based on the scan of the women’s breast/areola/nipple. For example, the slope of breast flange 502 may be designed to mimic the slope of the women’s breast based on the scan. Tube 506 is designed to be coupled a breast pump tube for applying suction in breast pump flange 500 responsive to pumping at the breast pump to pump milk from the breast. Bottle connector 508 is configured to be coupled to bottle 510 that receives and holds the milk pumped from the breast. Breast connector 508 may include a suitable coupling structures such as threads for screwing to corresponding threads on bottle 510.

Customized nipple portion 504 is sized and shaped to mimic a nursing woman’s anatomic nipple to reduce discomfort to the nursing woman. Similarly, breast flange 502 may be sized and shaped to mimic a nursing woman’s anatomic breast to reduce discomfort to the nursing woman. Breast pump flange 500 may be constructed to be coupled to a commercially available breast pump and/or bottle 510. For example, customized nipple portion 504 and breast flange 502 may be constructed based on the scanned information of the nursing woman’s nipple while the remaining portions of breast pump flange 500 may be constructed based on data stored in memory of the systems. Breast pump flange 500 is formed of a material commonly used in breast pump flanges such as a polymer and/or a material selected to mimic feel of the nursing woman’s nipple such as silicone, rubber, poly vinyl chloride, or latex, or combinations thereof.

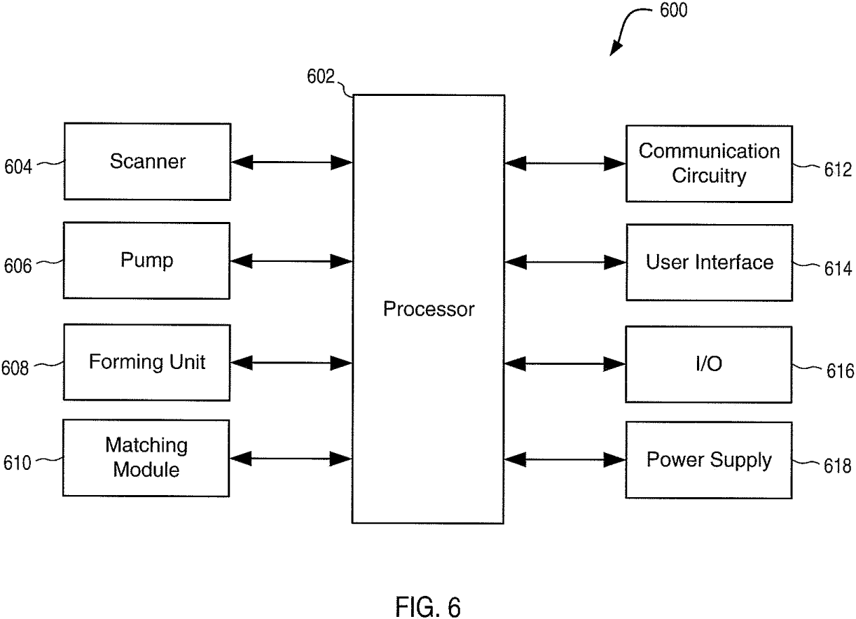

Referring to FIG. 6, a generalized schematic diagram of an exemplary system for processing information for a customized nipple in accordance with the principles of the present invention is now described. Customized nipple system 600 may include processor 602, scanner 604, pump 606, forming unit 608, matching module 610, communication circuitry 612, user interface 614, input and output circuitry (I/O) 616, and/or power supply 618. The components of customized nipple system 600 may be located in a single location or may be in more than one location. In one embodiment, the components of customized nipple system 600 are at least partially disposed within a common housing. In other aspects, multiple housings are used. Additionally, some or all of the components may be directly electrically coupled or may be electrically/electro-magnetically coupled via a network such as the Internet or Bluetooth.

Processor 602 may be electrically coupled to, and may control, the functional components of customized nipple system 600. Processor 602 may comprise one or more commercially available microcontroller units that may include programmable microprocessors, volatile memory, nonvolatile memory such as EEPROM for storing programming, and nonvolatile storage, e.g., Flash memory, for storing firmware and a log of user data, baby and/or nipple-related product catalogs, and/or algorithms for matching the scanned nipple to a preferred nipple from catalogs of nipples based on characteristic matching. The memory of processor 602 stores program instructions that, when executed by processor 602, cause processor 602 and the functional components of customized nipple system 600 to provide the functionality ascribed to them herein. Processor 602 is configured to be programmable such that programming data is stored in the memory of processor 602 or accessible via a network. As will be readily understood to one skilled in the art, while FIG. 6 is illustrated to show one processor, multiple processors may be utilized in a single location or multiple locations. Processor 602 may store user information, product catalog information, and/or matching algorithms in its memory or in memory accessible via a network, e.g., the Internet. For example, the memory may store information on a variety of commercially available baby bottles such that the customized nipple formed is compatible with the commercially available baby bottle. In this manner, a user may select the type of baby and/or nipple-related product for use with the customized nipple formed by the system. Additionally, or alternatively, the system could store various commercially available baby and/or nipple-related product selectable via a user interface and optionally visible in a vending machine-type format. In one embodiment, processor 602 is a processor in a computer such as a laptop, desktop, tablet, smartphone, smartwatch, etc.

Scanner 604 scans a woman’s nipple to generate data indicative of characteristics of the woman’s nipple for a customized nipple. Scanner 604 is preferably electrically coupled to processor 602 such that processor 602 receives data indicative of the characteristics of the woman’s nipple from the scan. Scanner 604 may scan the woman’s nipple in a noninvasive manner without the need to contact the nipple, breast, or skin for the scan. for example, using technologies such as LIDAR. Scanner 604 may take multiple photographs of the woman’s nipple that are combined by processor 602 to render a three-dimensional image of the woman’s nipple, for example, using the technologies associated with photogrammetry. Additionally, processor 602 may direct scanner 604 to initiate the scanning protocol responsive to user input received at the user interface. For example, once the woman’s nipple is in the desired state for the scan (e.g., a feeding state), the user may select “Begin Scan” at the user interface which causes scanner 604 to start scanning the nipple. The user interface may also provide instructions on photographing the nipple based on commands from processor 602. For example, the user interface may instruct the woman to take photographs of the woman’s nipple at predetermined viewing angles for rendering the three-dimensional image such as a top view, bottom view, front view, right side view, and/or left side view. Scanner 604 may utilize technology known in CAD/CAM fields for scanning the woman’s nipple and processing the scan for the customized nipple. A scanner from CADScan of Chester, UK may be used. In one embodiment, scanner 604 uses the camera of a computer such as a laptop, desktop, tablet, smartphone, smartwatch, etc. for scanning the woman’s nipple.

Pump 606 pumps the nipple such that the nipple transitions to the feeding state. For an accurate replica of a nursing woman’s nipple to avoid nipple confusion, it may be important that the customized nipple mimic the nipple in the feeding state. Pump 606 may be a commercially available breast pump. Pump 606 may be electrically coupled to processor 602 to direct pump 606 to initiate pumping responsive to user input received at the user interface. For example, the user may select “Begin Pumping” at the user interface which causes pump 606 to start pumping the nipple. Alternatively, the user may activate pump 606 when prompted by a message at the user interface. As an alternative to pump 606, the user interface may prompt the nursing woman to prepare the nipple, at which time the nursing mother has the baby latch on the nipple to feed to transition the nipple to the feeding state.

Forming unit 608 forms the customized nipple based on the data indicative of the characteristics of the woman’s nipple from the scan by scanner 604. In this manner, the customized nipple mimics the characteristics of the woman’s nipple. Additionally, forming unit 608 forms the customized nipple from a material selected, in embodiments such as baby bottle nipples, pacifiers, and prosthetic nipple, to mimic feel of the woman’s nipple such as silicone, rubber, poly vinyl chloride, or latex, or combinations thereof. Forming unit 608 may form the customized nipple of other material commonly used such as polymers for nipple shields and breast pump flanges. Forming unit 608 is preferably electrically coupled to processor 602 such that processor 602 causes forming unit 608 to form the customized nipple based on data indicative of the characteristics of the woman’s nipple from the scan. Additionally, processor 602 may direct forming unit 608 to initiate the forming protocol responsive to user input received at the user interface. Alternatively, processor 602 may direct forming unit 608 to automatically begin forming the customized nipple after an acceptable scan. Forming unit 608 may form the customized nipple based on matching the scanned nipple to a customized nipple based on a matching algorithm as described below. Forming unit 608 may form the customized nipple for sending to the user or for inventory replenishment. Forming unit 608 may be a three-dimensional printer and may print the customized nipple from the material selected to mimic feel of the woman’s nipple. For example, the 3D printer could print the customized nipple with silicone, rubber, poly vinyl chloride, or latex, or combinations thereof. Forming unit 608 may utilize CAD/CAM technology for forming the woman’s nipple. In one embodiment, forming unit 608 need not utilize injection molding techniques to form the customized nipple. For example, forming unit 608 may form a free-standing customized nipple without the need for a mold. In some embodiments, forming unit 608 forms the customized nipple using a mold (e.g., via injection molding) selected from a plurality of different molds with varying characteristics based on similarities between the characteristics of the selected mold and the characteristics of the scanned nipple.

In some embodiments, the customized nipple is scaled for the product. For example, if a baby is premature, a pacifier and/or bottle nipple based directly on the woman’s nipple may be too large for the premature baby. Thus, the customized nipple may be designed to mimic the woman’s nipple, but scaled down based on characteristics of the baby, e.g., weight, age, how premature the baby was born, etc. In this manner, processor 602 may receive user input on the characteristic(s) of the baby, determine a scaling factor based on the characteristic(s), and form/select a scaled version of the customized nipple using the scan and the scaling factor.

Matching module 610 matches a customized nipple to the woman from a plurality of customized nipples based on the data indicative of the characteristics of the woman’s nipple from the scan by scanner 604. In this manner, the customized nipple mimics the characteristics of the woman’s nipple. Matching module 610 executes algorithms to process data from the scan to compare the characteristics of the woman’s nipple to a plurality of other nipples’ characteristics. Matching module 610 may generate information for a customized nipple for the woman based on the comparison of the characteristics of the woman’s nipple with the other nipples’ characteristics such that the customized nipple mimics the characteristics of the woman’s nipple. In some embodiments, matching module 610 is executed by processor 602 such that processor 602 executed instructions to match a customized nipple based on data indicative of the characteristics of the woman’s nipple from the scan. In some embodiments, forming unit 608 forms the customized nipple using a mold selected from a plurality of different molds based on similarities between the characteristics of the selected mold and the characteristics of the scanned nipple. In some embodiments, matching module 610 is incorporated into an “app” that is stored on a computing device (e.g., laptop, desktop, tablet, smartphone, smartwatch, etc.).

Examples of characteristics of the woman’s nipple that may be compared via matching module 610 to the plurality of other nipples’ characteristics may include: diameter of the nipple, diameter of an areola, depth/length of the nipple, depth/length of the areola, depth/length of the nipple plus the areola, color of the nipple, color of the areola, slope of the nipple, slope of the areola, texture of the nipple, or texture of the areola, or any combination thereof. In this manner, the woman’s scanned nipple may be matched to a preferred nipple from a catalog of nipples with varying characteristics based on similarities of the characteristics of the scanned nipple to the preferred nipple. The algorithms may utilize lookup tables to match scanned nipple characteristics to the corresponding characteristics of the other nipples. Additionally or alternatively, the algorithms may score each characteristic and compare the scores to the plurality of other nipples to determine the match based on classification algorithms utilizing machine learning technology. The lookup tables and/or scores may be stored in memory with information on each characteristic of the plurality of other nipples for the comparison. For example, a first template may have a ½ inch nipple diameter, ½ inch nipple length, and a smooth nipple texture, a second template may have a ½ inch nipple diameter, ½ inch nipple length, and a rough nipple texture, and a third template may have a ½ inch nipple diameter, ¾ inch nipple length, and a smooth nipple texture. If the woman’s scan indicates that the woman’s nipple is ½ inch nipple diameter, ½ inch nipple length, and a smooth nipple texture, the scan would be matched to the first template and the woman would be sent a product with a customized nipple based on the first template so as to mimic the woman’s nipple. The characteristics may be scored using scaling for the characteristic. For example, if a score is between 0 and 1 for each characteristic, a score may be assigned in a corresponding manner, such as 0.1 for ⅛ inch nipple diameter, 0.2 for ¼ inch nipple diameter, 0.3 for ⅜ inch nipple diameter, etc. to 1 for 1 ¼ inch nipple diameter. Increments could be used in suitable levels such as 0.15 for 3/16 inch, etc. The characteristics may be weighted based on the significance of the characteristic for the product. For example, when the diameter and depth of the nipple are the most significant characteristics for a product, the diameter and depth of the nipple would be weighted with higher values than other characteristics for ensuring the match with the preferred nipple from the plurality of available nipples.

In some embodiments, matching module 610 compares the scanned nipple to the plurality of other nipples to match to the preferred nipple using algorithms stored locally of the computing device. This may assist with security of the nipple scans, as the data on the nipple scan need not necessarily be transferred from the computing device via a network. Instead, information on the preferred nipple selected from the match could be transferred via the network for sending the woman formed preferred nipple, without actually sending image data of the scans themselves. Additionally or alternatively, formation unit 608 may form the nipple locally based on the selected preferred nipple from the match without the need to transfer data on the scan via a network.

Once the scanned nipple is matched to the selected preferred nipple, the woman should receive the selected product(s) (e.g., baby bottle nipple(s), pacifier(s), prosthetic nipple(s), nipple shield(s), attachment(s) for supplemental nursing system(s), and/or breast pump flange(s)) incorporating the selected preferred nipple. The selected preferred nipple may be formed by printing, with a mold, etc., as described above and may be selected from an inventory of the selected preferred nipples.

Communication circuitry 612 is configured to transfer information, such as data indicative of the characteristics of the woman’s nipple, user account information, the matched customized nipple, and/or selected product information, to a remote location such as a remote server via a network such as the Internet. Communication circuitry 612 may include one or more wireless chipsets; e.g., WiFi, Bluetooth, and/or cellular; configured for wireless communication over a network such as the Internet, a local network, or a telephone network using techniques known in the art. In this manner, data associated with the nipple scan may be transferred and stored via communication circuitry 612. Communication circuitry 612 may be wireless circuitry in a computer such as a laptop, desktop, tablet, smartphone, smartwatch, etc. Communication circuitry 612 is preferably electrically coupled to processor 602 such that processor 602 causes communication circuitry 612 to transfer information remotely responsive to user input received at the user interface. In one embodiment, processor 602 runs a routine to encrypt information transmitted from communication circuitry as such information may be sensitive for a user. Responsive to user input received at the user interface, processor 602 may cause communication circuitry 612 to transfer data indicative of the characteristics of the nursing woman’s nipple such that the customized nipple is formed remotely with forming unit 608. Additionally, a user may set up an account with a secure login such that the user may log in to the account more than once to order additional customized nipples based on data from a new scan or data from a previous scan stored in memory at processor 602 or remotely.

User interface 614 is configured to receive user input and, optionally, to display information to the user. User interface 614 may include buttons for receiving user input and a display, e.g., touchscreen, for displaying information to the user. As will be readily apparent to one skilled in the art, user interface 614 is not limited thereto and may use one or more of a touch screen, a keypad, a microphone, a speaker, a trackball, or the like. A user may, for example, provide user input by pressing the touchscreen to cause processor 602 to cause scanner 604 to begin the scanning protocol. Then, the scanned nipple may be received and the display of user interface 614 may display the scanned nipple. The display of user interface 614 may further display how the formed customized nipple will look in the selected baby/nipple/nursing product such as in the selected bottle, pacifier, prosthetic nipple, nipple shield, attachment for supplemental nursing systems, or breast pump flange.

Input and output circuitry (I/O) 616 may include ports for data communication such as wired communication with a computer/mobile device and/or ports for receiving removable memory, e.g., SD card, upon which program instructions or data related to scanning and/or forming use may be stored. In one embodiment, I/O 616 comprises a port, and corresponding circuitry, for accepting a cable(s) to electrically couple processor 602 to scanner 604, pump 606, and/or forming unit 608.

Power supply 618 powers one or more of the electrical components of customized nipple system 600, and may be a primary cell or battery, a secondary (rechargeable) cell or battery or a combination of both. Alternatively, power supply 618 may be a port (e.g., USB port) to allow customized nipple system 600 to be plugged into a conventional wall socket for powering components. In one embodiment, power supply 618 comprises the rechargeable battery of the computer.

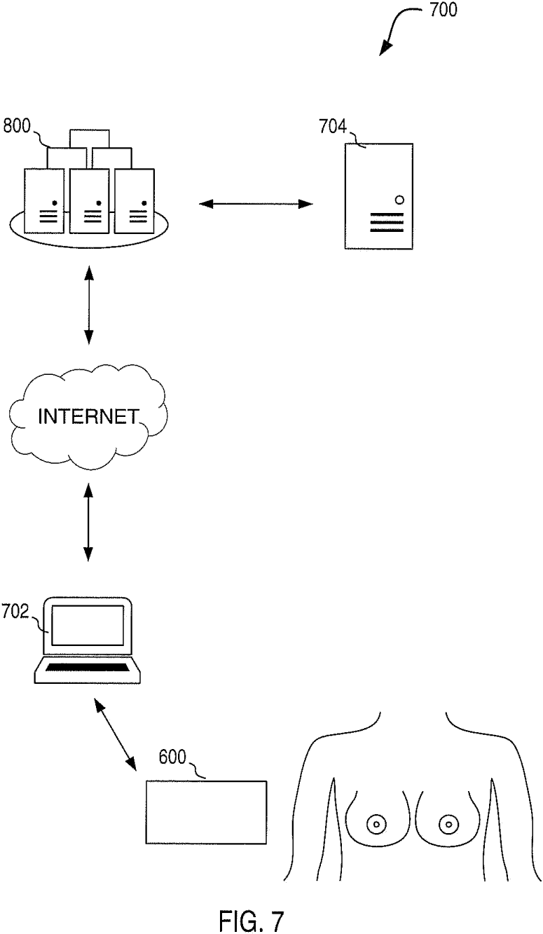

Referring now to FIG. 7, exemplary components for executing nipple system 700 utilizing further scan processing techniques, such as machine leaming/artificial intelligence, are illustrated. FIG. 7 shows customized nipple system 600, system device 702, and server 800, as well as drive 704. Customized nipple system 600 is described above. In some embodiments, customized nipple system 600 communicates to servers executing machine learning technology to identify characteristics associated with a desired outcome such as optimal breast feeding accessories and/or enhanced mimicking of the anatomic nipple by the customized nipple.

Optionally, customized nipple system 600 may communicate locally with system device 702 for communicating with a network such as the Internet. As will be readily understood, when customized nipple system 600 incorporates suitable technology for communicating with the network, system device 702 is not needed. System device 702 is preferably one or more computing devices (e.g., laptop, desktop, tablet, smartphone, smartwatch, etc.) having hardware and software components known in the art.

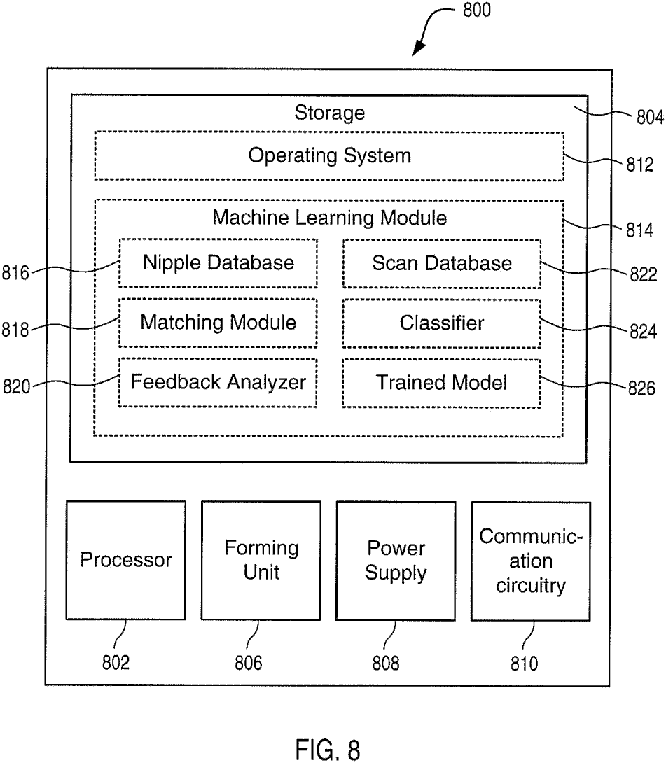

Server 800 is preferably one or more servers having the components described below with reference to FIG. 8 and the functionality described herein. Server 8 preferably has processing power superior to system 600 and system device 702 such that server 800 can process and analyze a significant number of nipple scans and further store information associated with the nipple scans, such as datasets of scans from many different women classified by characteristic. As will be readily apparent to one skilled in the art, server 800 may include a plurality of servers located in a common physical location or in different physical locations. In a preferred embodiment, server 800 is located in a different, remote location (e.g., on the cloud) than system 600 and/or system device 702, although server 800, system 600, and/or system device 702 may be located in a common location (e.g., on a local area network (LAN)).

Server 800 may optionally communicate with drive 704 which may be one or more drives having memory dedicated to storing digital information unique to a certain patient, scan, product, and/or analysis. For example, drive 704 may include, but is not limited to, volatile (e.g. random-access memory (RAM)), non-volatile (e.g. read-only memory (ROM)), flash memory, or any combination thereof. Drive 704 may be incorporated into server 800 or may be separate and distinct from server 800 and may communicate with server 800 over any well-known wireless or wired connection.

Referring now to FIG. 8, hardware and software components of server 800 may include processor 802, storage 804, forming unit 806, power supply 808, and/or communication circuitry 810. Processor 802 may be one or more processors configured to run operating system 812 and machine learning module 814 and perform the tasks and operations of server 800 set forth herein. Given the volume of data and processing tasks assigned to processor 802, it is understood that processor 802 preferably has superior processing power compared to processor 602.

Processor 802 preferably incorporates memory that may include, but is not limited to, volatile (e.g. random-access memory (RAM)), non-volatile (e.g. read-only memory (ROM)), flash memory, or any combination thereof. Storage 804 may include, but is not limited to, removable and/or non-removable storage such as, for example, magnetic disks, optical disks, or tape. Forming unit 806 may be designed in the same manner as forming unit 608 described above. In some embodiments, forming unit 806 is located in the same location as server 800, such as a commercial building/warehouse. Power supply 808 is any power source known for powering server-type equipment. Communication circuitry 810 may be designed in the same manner as communication circuitry 612 described above.

Operating system 812 and machine learning module 814 may be stored in storage 804 and executed on processor 802. Operating system 812 may be suitable for controlling general operation of server 800. Machine learning module 814 may be a software application and/or software modules having one or more sets of instructions. Machine learning module 814 may facilitate and oversee the processing and analysis of scanned nipple data received from system 600 and otherwise may be suitable for performing the operations of server 800 set forth herein.

Machine learning module 814 may include several sub-modules and/or applications including, but not limited to, nipple database 816, matching module 818, feedback analyzer 820, scan database 822, classifier 824, and/or trained model 826. Each sub-module and/or application may be a separate software application and/or module having one or more sets of instructions.

Nipple database 816 may be a collection of nipples with differing characteristics from one another. For example, nipple database 816 may store information on the various nipple templates described above with respect to matching module 610. In some embodiments, due to the superior processing and storage capabilities of server 800, it may be beneficial to store nipple database 816 at server 800.

Matching module 818 is designed to match the woman’s scanned nipple with the closest nipple template in nipple database 816 such that the selected nipple template, and thereby the customized nipple formed therefrom, mimics characteristics of the woman’s anatomical nipple. Matching module 818 may operate in the manner of matching module 610 described above. In some embodiments, due to the superior processing and storage capabilities of server 800, it may be beneficial to operate matching module 818 at server 800.

Feedback analyzer 820 receives user feedback on the customized nipple received by the user and analyzes the feedback. The feedback may be used to train matching module, classifier, and/or trained model. Examples of the types of feedback that may be received and analyzed by feedback analyzer are described below.

Scan database 822 may be a collection of scans of different woman’s nipples and information associated therewith. Information in scan database 822 (and other sensitive information described herein) is stored in a secure manner such as via encryption, as such information may be sensitive and to comply with regulations for handling patient data.

Classifier 824 receives data associated with scans of women’s nipples and classifies characteristics of the data. The characteristics may be predetermined and stored as classified datasets of the plurality of other nipples’ characteristics. For example, classifier 824 may receive outputs from scan database 822 and classify each nipple scan of the plurality of scans. The characteristics may be identified by machine classification. Additionally or alternatively, the characteristics to be classified may be predetermined. Examples of characteristics of the woman’s nipple that may be classified include: diameter of the nipple, diameter of an areola, depth/length of the nipple, depth/length of the areola, depth/length of the nipple plus the areola, color of the nipple, color of the areola, slope of the nipple, slope of the areola, texture of the nipple, or texture of the areola, or any combination thereof. Classifier 824 may classify images from the scan at the pixel level and/or at specific regions of interest utilizing the trained algorithms. Classifier 824 may run one or more neural networks to perform classification. Classifier 824 may represent the classified characteristics for each scan using at least one vector. For example, the output of a classification neural network maybe the vector [0.60; 0.70; 0.00] with the corresponding labels for each element of the vector. The scores may be between 0 and 1. For each vector, the score may be compared to a threshold (e.g., 0.5 score) and if the score exceeds the threshold, the characteristic may be labeled accordingly. It is understood that the threshold may be preprogrammed and/or selected by the user and may be modified to provide varying degrees of sensitivity and specificity. The vector(s) may be inputted into at least one neural network to generate at least one matrix for providing the customized nipple for the woman with characteristics that most closely match the woman’s anatomical nipple. The at least one matrix may be processed by one or more neural networks to generate information used to form the most preferred baby/nipple/nursing product.

Classifier 824 may apply unsupervised classification technologies to 2D and/or 3D anatomical data to identify pertinent nipple/breast/nursing characteristics and create appropriate standardized custom classes for preferred baby/nipple/nursing products that perform better than conventional products. In addition or alternatively, classifier 824 may apply supervised classification technologies to 2D and/or 3D anatomical data to identify pertinent nipple/breast/nursing characteristics and create appropriate standardized custom classes for preferred baby/nipple/nursing products that perform better than conventional products. Classifier 824 may apply trained classification algorithms to new 2D and/or 3D anatomical data to appropriately classify the new data into one of the identified standardized custom classes. Further, new 2D and/or 3D anatomical data may be used to improve the trained classification algorithms. The supervised and/or unsupervised classification technologies may be used to associate nipple/breast/nursing characteristics with outcomes, such as improved nursing outcomes, improved pacifying outcomes, and/or improved aesthetics, as compared to conventional techniques

The outputs of classifier 824 may be used for training to generate trained model 826. Trained model may include classified datasets associated with characteristics of many nipples from many sources. As more nipples are scanned, more nipples are classified, thereby further training trained model 826. As the amounts of data increase, it is expected that trained model 826 will improve.

Server 800 may also optionally run a graphics library, other operating systems, and/or any other application programs. It of course is understood that server 800 may include additional or fewer components than those illustrated in FIG. 8 and may include more than one of each type of component.

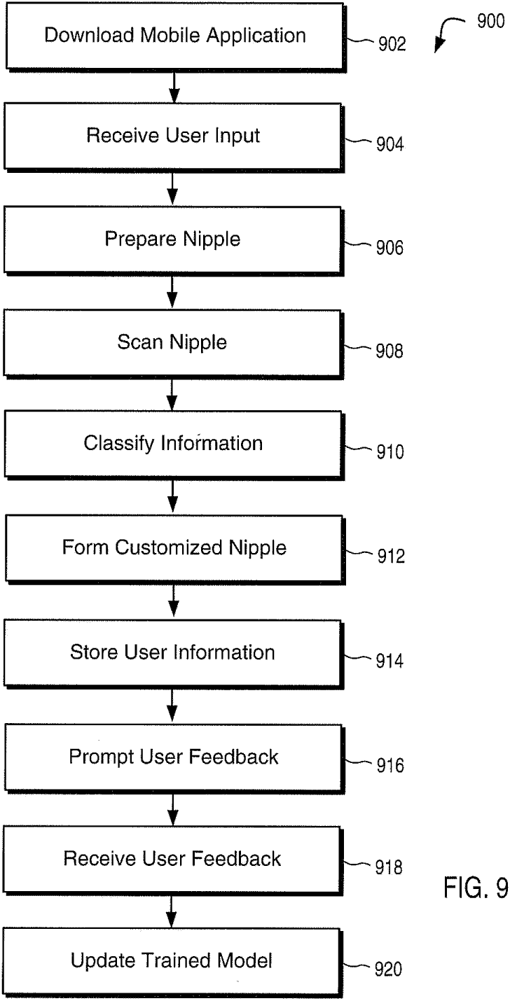

Referring now to FIG. 9, an exemplary method for processing a customized nipple in accordance with some embodiments of the present invention is shown. As will be apparent to one of ordinary skill in the art, method 900 may be embodied in program instructions stored on the memory of a computer that, when executed by one or more processors, cause the computer to provide the functionality ascribed to them herein. The program instructions may be software downloaded onto the memory of the computer. For example, a mobile application may be downloaded onto non-transitory computer readable media. Steps in method 900 may be performed in any order and the steps are optional such that not all steps need be performed.

At 902, a mobile application is downloaded onto the computer. The mobile application may be a dedicated application or “app” and may be downloaded from an online store such as iTunes™ (Apple, Inc., Cupertino, CA), the App Store (Apple, Inc.), Google™ Play (Google, Inc., Mountain View, CA), the Android™ Marketplace (Google, Inc.), Windows™ Phone Store (Microsoft Corp., Redmond, WA), or BlackBerry™ World (BlackBerry, Waterloo, Ontario, Canada). Preferably, the mobile application need only be downloaded once — although updates may be downloaded — and some or all of the remaining portions of method 900 may be repeated without the need to repeat 902. As will be readily apparent to one skilled in the art, the mobile application is not necessary and a user could instead login via a different mechanism such as a webpage. For example, a customized webpage may be used or a previously available webpage such as amazon.com.

At 904, user input is received at user interface 614. For example, a user (e.g., the woman, a family member, a doctor, nurse, lactation consultant, etc.) may create or log into a personal account. The user may select the type of product to be formed. For example, the user may select a customized nipple for a type and/or size of commercially available bottle from a catalog of commercially available bottle types and/or sizes stored in memory at the processor or stored remotely and accessible via a network such as the Internet. The user could also select the size of the bottle and the size of the hole in the customized nipple. If the user selects a pacifier, the user could select the type of base and/or handle for the pacifier from a catalog of commercially available pacifier base and/or handle types stored in memory at the processor or stored remotely and accessible via a network such as the Internet. The user could select a nipple shield, a prosthetic nipple, attachment for supplemental nursing systems, and/or a breast pump flange. The user may select a customized nipple for a type of commercially available breast pump from a catalog of commercially available breast pumps stored in memory at the processor or stored remotely and accessible via a network such as the Internet such that the breast pump flange with the customized nipple is compliant with the breast pump.

Additional user input may be collected responsive to surveys taken by the users. Such user input may be stored for evaluating the efficacy of the product. Example survey questions include:
Gestational age of child at birth
Characteristics of child anatomy
Head size of child
Does the child have a cleft palate?
Is the child considered tongue-tied?
Age of child at first use of other pacifier
Age of child at first use of other bottle nipple
Age of child at first use of other nipple shield
Age of child at first use of customized pacifier
Age of child at first use of customized bottle nipple
Age of child at first use of customized nipple shield
Age of child at time of survey
Ease with which child latched onto breast at birth
   1 being impossible, 10 being extremely easy, 0 not applicable
Ease with which child latched onto breast at first time of use of other pacifier
   1 being impossible, 10 being extremely easy, 0 not applicable
Ease with which child latched onto breast at first time of use of other bottle nipple
   1 being impossible, 10 being extremely easy, 0 not applicable
Ease with which child latched onto breast at first time of use of other nipple shield
   1 being impossible, 10 being extremely easy, 0 not applicable
The best latch onto your breast your child ever achieved after use of other pacifier
   1 being impossible, 10 being extremely easy, 0 not applicable
The best latch onto your breast your child ever achieved after use of other bottle nipple
   1 being impossible, 10 being extremely easy, 0 not applicable
The best latch onto your breast your child ever achieved after use of other nipple shield
   1 being impossible, 10 being extremely easy, 0 not applicable
The best latch onto your breast your child ever achieved after use of other pacifier
   1 being impossible, 10 being extremely easy, 0 not applicable
The best latch onto your breast your child ever achieved after use of customized pacifier
   1 being impossible, 10 being extremely easy, 0 not applicable
The best latch onto your breast your child ever achieved after use of customized bottle nipple
   1 being impossible, 10 being extremely easy, 0 not applicable
The best latch onto your breast your child ever achieved after use of customized nipple shield
   1 being impossible, 10 being extremely easy, 0 not applicable
The customized nipple shield fit onto my nipple better than the other nipple shield
   1 strongly disagree, 10 strongly agree, 0 I did not use the customized nipple shield

At 906, the woman’s nipple is prepared for the scan. Preferably, the nipple is in the feeding state for the scan. To achieve the feeding state, the breast may be pumped with pump 606. Alternatively, the baby may feed on the nursing woman’s nipple. User interface 614 may prompt the nursing woman to prepare the nipple such as through a message instructing the nursing woman to begin pumping or to begin a feeding session.

At 908, the woman’s nipple is scanned, e.g., via scanner 604, to generate data indicative of characteristics of the woman’s nipple for the customized nipple. User interface 614 may permit a user to initiate the scanning protocol, e.g., by pressing a button or the touchscreen to begin the scan of the nipple. The scanned nipple may be displayed on user interface 614 as well as the expected look of the formed baby/nipple/nursing product with the customized nipple.

At 910, information from the scan may be classified, for example using classifier 824 as described above.

At 912, the customized nipple is formed. Preferably, the customized nipple is formed based on the data indicative of the characteristics of the woman’s nipple from the scan such that the customized nipple mimics the characteristics of the woman’s nipple. Forming the customized nipple may include providing the customized nipple based on the matching algorithms described herein. If the selected product is a pacifier, the entire pacifier may be formed including the pacifier base, handle, and customized nipple or the customized nipple may be formed on a pre-stored pacifier base with a handle. If the selected product is a bottle, the entire bottle may be formed including the container, cap, and customized nipple or the customized nipple may be formed. The remaining portions of the bottle may be provided from the system or user may use separately purchased bottled parts. In one embodiment, the woman’s nipple is scanned and the customized nipple is formed in the same location. Accordingly, the customized nipple may be formed in a relatively fast amount of time (e.g., less than one hour). The customized nipple may be formed by printing the customized nipple, e.g., with a three-dimensional (3D) printer. The customized nipple may be formed from a mold selected based on the matching algorithms described herein to closely match the scanned nipple. In such an embodiment, a plurality of molds are available and the matching algorithms match the scanned nipple with the closest mold (e.g., via the nipple templates) based on the characteristics analyzed from the scan. Preferably, the customized nipple is formed from a material selected to mimic feel of the nursing woman’s nipple such as silicone, rubber, poly vinyl chloride, or latex, or combinations thereof or a material commonly used for the product such as a polymer for a nipple shield or breast pump flange.

At 914, the user information is stored. For example, user account information may be stored locally or remotely including user identification information. As such, a user may log back into the account with a secure login to order additional customized nipples based on data from a new scan or data from a previous scan stored in memory at processor 902 or stored in memory remotely, e.g., remote server, cloud.

At 916, user feedback on the product is requested. For example, the user feedback may be requested via a prompt on the app with an opportunity to answer further questions on the product. Example follow-up questions for the survey include:
Total length of time you breast fed your child
Prior to using a customized pacifier, I was ready to quit breast feeding my child
   1 strongly disagree, 10 strongly agree, 0 I did not use the customized pacifier
Prior to using a customized bottle nipple, I was ready to quit breast feeding my child
   1 strongly disagree, 10 strongly agree, 0 I did not use the customized bottle nipple
Prior to using a customized nipple shield, I was ready to quit breast feeding my child
   1 strongly disagree, 10 strongly agree, 0 I did not use the customized nipple shield
The use of a customized pacifier allowed me to breast feed by child for longer than I would have if I had not used a customized pacifier
   1 strongly disagree, 10 strongly agree, 0 I did not use the customized pacifier
The use of a customized bottle nipple allowed me to breast feed by child for longer than I would have if I had not used a customized bottle nipple
   1 strongly disagree, 10 strongly agree, 0 I did not use the customized bottle nipple
The use of a customized nipple shield allowed me to breast feed by child for longer than I would have if I had not used a customized nipple shield
   1 strongly disagree, 10 strongly agree, 0 I did not use the customized nipple shield
The use of a customized pacifier improved my child’s ability to transition from breast to bottle
   1 strongly disagree, 10 strongly agree, 0 I did not use the customized pacifier
The use of a customized bottle nipple improved my child’s ability to transition from breast to bottle
   1 strongly disagree, 10 strongly agree, 0 I did not use the customized bottle nipple
The use of a customized nipple shield improved my child’s ability to transition from breast to bottle
   1 strongly disagree, 10 strongly agree, 0 I did not use the customized nipple shield

At 918, user feedback is received. For example, the scores selected by the user may be received and compared to scores from the initial survey. Based on the survey results, characteristics associated with product efficacy may be analyzed.

At 920, the user feedback may be used to train the trained model. For example, information associated with the scores may be inputted into trained model 828 to further train the model.

Customized nipple system 600 is well suited for in-home use, at a doctor’s office, at a hospital, with a lactation consultant, etc., which could each be in a single room. Customized nipple system 600 could be in mobile form such as on a mobile cart or in a lockable booth located at a commercial area such as a mall.

While various illustrative embodiments of the invention are described above, it will be apparent to one skilled in the art that various changes and modifications may be made therein without departing from the invention. The appended claims are intended to cover all such changes and modifications that fall within the true scope of the apparatus and methods of the present invention.

What is claimed:

1. A method of generating information associated with a customized nipple for use in a baby or nursing or nipple-related product, the method comprising:
scanning a woman’s nipple to generate data indicative of characteristics of the woman’s nipple;
processing the data to compare the characteristics of the woman’s nipple to a plurality of other nipples’ characteristics; and
generating information for a customized nipple for the woman based on the comparison of the characteristics of the woman’s nipple with the other nipples’ characteristics such that the customized nipple mimics the characteristics of the woman’s nipple.

2. The method of claim 1, wherein the characteristics of the woman’s nipple comprise at least one of: diameter of the nipple, diameter of an areola, depth/length of the nipple, depth/length of the areola, depth/length of the nipple plus the areola, color of the nipple, color of the areola, slope of the nipple, slope of the areola, texture of the nipple, or texture of the areola, or any combination thereof.

3. The method of claim 1, wherein processing the data comprising generating a score for each of the characteristics of the woman’s nipple to compare to corresponding scores stored for the plurality of other nipples’ characteristics.

4. The method of claim 3, wherein the scores are weighted by characteristic such that scores of a more significant characteristic receive a higher weighted value than less significant characteristics.

5. The method of claim 3, further comprising representing the scores for each of the characteristics as at least one vector.

6. The method of claim 5, wherein the at least one vector is inputted into at least one neural network to generate at least one matrix for the customized nipple for the woman.

7. The method of claim 6, wherein the at least one neural network utilizes machine learning technology for the customized nipple of the woman.

8. The method of claim 1, wherein processing the data comprising classifying characteristics of the woman’s nipple.

9. The method of claim 8, wherein the characteristics are identified by machine classification.

10. The method of claim 8, wherein the characteristics are predetermined based on a classified dataset of the plurality of other nipples’ characteristics.

11. The method of claim 8, further comprising training a trained model of classified datasets associated with characteristics of the plurality of other nipples using the classified characteristics of the woman’s nipple.

12. The method of claim 1, wherein the method is performed using an app downloaded to a computing device.

13. The method of claim 1, further comprising sending the data to a remote computing system, and
wherein processing the data and generating the information occur at the remote computing system.

14. The method of claim 1, further comprising sending at least one product incorporating the customized nipple to the woman.

15. The method of claim 14, wherein the at least one product comprises at least one baby bottle nipple, pacifier, prosthetic nipple, nipple shield, attachment for supplemental nursing system, or breast pump flange, or any combination thereof.

16. The method of claim 1, wherein generating the information comprises selecting the customized nipple for the woman from a plurality of customized nipples with predetermined characteristics, each of the plurality of customized nipples having at least one different predetermined characteristic from another.

17. The method of claim 16, wherein selecting the customized nipple comprises executing a matching algorithm to match the woman’s nipple to the customized nipple.

18. The method of claim 1, further comprising forming the customized nipple.

19. The method of claim 18, wherein forming the customized nipple comprises printing the customized nipple.

20. The method of claim 1, wherein the customized nipple is formed of a material selected to mimic feel of the woman’s nipple.

21. The method of claim 1, further comprising scaling the customized nipple based on user input associated with information on the woman’s baby.

22. A product bottle comprising the customized nipple of claim 1, the product selected from a bottle, a pacifier, a nipple shield, a prosthetic nipple, a breast pump flange, or a component for a supplemental nursing system.

23. A non-transitory computer readable medium comprising instructions that, when executed by at least one processor, cause the at least one processor to:
receive data indicative of characteristics of a woman’s nipple based on a scan of the woman’s nipple;
process the data to compare the characteristics of the woman’s nipple to a plurality of other nipples’ characteristics; and
generate information for a customized nipple for the woman based on the comparison of the characteristics of the woman’s nipple with the other nipples’ characteristics such that the customized nipple mimics the characteristics of the woman’s nipple.

24. A system for generating information associated with a customized nipple for use in a baby or nursing or nipple-related product, the system comprising at least one processor configured to execute instructions to cause the at least one processor to:
receive data indicative of characteristics of a woman’s nipple based on a scan of the woman’s nipple;
process the data to compare the characteristics of the woman’s nipple to a plurality of other nipples’ characteristics; and
generate information for a customized nipple for the woman based on the comparison of the characteristics of the woman’s nipple with the other nipples’ characteristics such that the customized nipple mimics the characteristics of the woman’s nipple.
